(19) United States
(12) Patent Application Publication
(10) Pub. No.: US 2026/0137941 A1
(43) Pub. Date: May 21, 2026

(54) SYSTEMS AND METHODS FOR SELECTIVE HEATINDUCED NEUROMODULATION

(71) Applicants: Case Western Reserve University, Cleveland, OH (US); VANDERBILT UNIVERSITY, Nashville, TN (US)

(72) Inventors: Michael W. JENKINS, Cleveland, OH (US); Junqi ZHUO, Cleveland, OH (US); Hillel J. CHIEL, Cleveland, OH (US); Eric Duco JANSEN, Nashville, TN (US)

(21) Appl. No.: 18/704,612

(22) PCT Filed: Aug. 26, 2022
(86) PCT No.: PCT/US2022/041658

§ 371 Date: Apr. 25, 2024

Related U.S. Application Data
(60) Provisional application No. 63/272,800, filed on Oct. 28, 2021.

(51) Int. Cl. A61N 1/36; A61N 1/05; A61N 1/40

(52) U.S. Cl. CPC A61N 1/36071; A61N 1/0556; A61N 1/36139; A61N 1/403

(57) ABSTRACT

Controlled heating can be used to achieve selective nerve block of certain neural fibers. As an example, the selective heat-induced neural modulation can be used for pain management. A controller can determine an amount of energy to be delivered to a heat delivery component of a neural prosthetic device for conversion to a heat signal and application to a nerve to block neural conduction in at least a portion of the nerve. The amount of energy to be delivered is determined based on a thermal dose profile. The controller can receive a signal comprising a temperature of the nerve when the heat signal is applied to the nerve; and alter the amount of energy received by the heat delivery component based on the temperature of the nerve so the amount of energy follows the thermal dose profile, and the temperature of the nerve remains within an allowable range.

### RELATED APPLICATION

This application claims priority to U.S. Provisional Application Ser. No. 63/272,800, filed Oct. 28, 2021, entitled “SYSTEMS AND METHODS FOR SELECTIVE HEAT BLOCK IN NERVE”, the entirety of which is hereby incorporated by reference for all purposes.

### TECHNICAL FIELD

The present disclosure relates generally to neuromodulation and, more specifically, to systems and methods for applying controlled heating to achieve selective heat-induced neuromodulation.

### BACKGROUND

Small-diameter nerve fibers carry various sensory signals that are critical for the homeostasis of vital physiological conditions. Damage to afferent sensory nerve fibers, for example from injuries or other pathological processes, may trigger neuropathic pain. Neuropathy affects approximately 7-10 percent of the general population. Several treatments have been used for neuropathy with varying levels of success. Pharmaceutical treatments, especially those involving opioids, have long been used as the primary means for pain management, but suffer from lack of selectivity as well as the risk of addiction and other serious side effects. Surgical treatments can cut or ablate the disrupted nerve to block signals related to the physiological conditions, but risk permanently damaging other functions (e.g., motor functions) of the nerve. Traditional electrode-based neuromodulation treatments do have the ability to induce neural block. In fact, small diameter fibers can be blocked with electrode-based neuromodulation treatments, but the small fibers are not blocked preferentially because large diameter axons must be blocked first due to size constraints of electrical stimulation. Accordingly, electrical-based neuromodulation can achieve desired block of small diameter fibers but can also cause as many problems as it solves by simultaneously blocking larger diameter axons (e.g., motor axons).

Selective inhibition of small-diameter axons is a critical and unmet medical need with the potential to treat various diseases (e.g., pain, obesity, hypertension, etc.) that are difficult to treat using traditional methods. Heat can be used to achieve such selective inhibition. In fact, infrared neural inhibition (INI) has been shown to achieve size selectivity, as well as spatial selectivity, via application of heat. However, INI is not easy to implement within a living subject. For example, INI is not energy efficient, can require excess heating that can cause tissue damage to reach certain block thresholds, and has several implantation issues.

### SUMMARY

Described herein is a treatment that can apply controlled heating to achieve selective inhibition (e.g., of small-diameter fibers, of fibers in different locations, etc.). The selective inhibition can preferentially block small-diameter fibers or preferentially block in a spatial location with controlled temperature adjustments.

An aspect of the present disclosure is a system that can apply heat-based neuromodulation selectively to certain neural fibers (e.g., small diameter fibers) to establish a conduction block in the certain neural fibers. The system includes a controller device configured to deliver an amount of energy to a neural prosthetic device; and the neural prosthetic device configured to be positioned near at least a part of a nerve of a patient. The neural prosthetic device includes a heat delivery component configured to convert the amount of energy to an amount of heat and to deliver the amount of heat to the part of the nerve to block conduction in at least a portion of the nerve; and a temperature sensor configured to record a temperature of the nerve. The controller device is further configured to: store a thermal dose profile for the patient and information regarding a predetermined heat boundary for the nerve; and alter the amount of energy sent to the neural prosthetic device based on the temperature of the nerve to maintain the temperature of the nerve within the thermal dose protocol of the patient and the predetermined heat boundary for the nerve.

Another aspect of the present disclosure is a method for selective heat-induced neuromodulation to establish a conduction block in certain neural fibers (e.g., small diameter neural fibers. As an example, the method can be performed by a controller that includes at least a processor. The method includes determining an amount of energy to be delivered to a heat delivery component of a neural prosthetic device for conversion to a heat signal and application to a nerve to block conduction in at least a portion of the nerve, wherein the amount of energy to be delivered is determined based on a thermal dose profile stored by the controller; receiving a signal comprising a temperature of the nerve when the heat signal is applied to the nerve; and altering the amount of energy received by the heat delivery component based on the temperature of the nerve so the amount of energy follows the thermal dose protocol and the temperature of the nerve remains within an allowable range.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a representation of a system that can apply controlled heating to achieve selective inhibition;

FIG. 2 is a representation of the controller device of FIG. 1 and the communication between the controller device and other elements of the system;

FIG. 3 is a representation of the neural prosthetic device of FIG. 1 and the communication between the neural prosthetic device and the controller device;

FIG. 4 is an example of the neural prosthetic device that can be used in the system of FIG. 1;

FIG. 5 is a cut section of the example neural prosthetic device of FIG. 4;

FIG. 6 is a process flow diagram showing a method of applying controlled heating to achieve selective inhibition;

FIG. 7 shows schematics and image of an experimental heating cuff design and the experimental setup;

FIG. 8 is a graphical representation of change in temperature recordings of an experimental heating trial;

FIG. 9 shows representative compound action potential (CAP) and corresponding normalized rectified area under the curve (RAUC) for small- and large-diameter fibers;

FIG. 10 is a graphical representation of the inhibition selectivity index of the slow conducting small-diameter fibers for IR neural inhibition and resistive heating;

FIG. 11 is graphical representations of the comparative efficiency of the power applied at the neural interface and the total electrical power used for IR inhibition versus resistive heating; and

FIG. 12 is results of simulated radial temperature distributions and inhibition probability under different temperature elevations.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “neuromodulation” refers to the alteration of nerve activity through targeted delivery of a stimulus to one or more specific neural sites in a patient's body. Alteration of nerve activity can include blocking or enhancing conduction in a nerve. For example, a block can be a partial block or a full block of one or more portions of the nerve.

As used herein, the term “stimulus” refers to something that evokes a specific alteration of nerve activity when delivered to one or more specific neural sites in a patient's body. The stimulus can include a heat signal and may also include an electrical signal, a chemical signal, or the like.

As used herein, the term “neural prosthetic device” refers to an implanted instrument that is configured to deliver a stimulus to one or more specific neural sites in a patient's body for neuromodulation.

As used herein, the term “heat delivery component” refers to at least a portion of a neural prosthetic device that delivers a heat signal stimulus to at least a portion of nerve fibers within one or more specific neural sites in a patient's body. The heat delivery component can include, for example, a resistive heating element, ultrasound heating element, radio-frequency heating element, or the like.

As used herein, the term “resistive heating element” refers to a conductor that is undergoing the process of resistive (or Joule) heating where heat is produced by the passage of an electric current through the conductor.

As used herein, the term “temperature sensor” refers to a device for measuring and/or recording temperature in an environment. Examples of temperature sensors include, but are not limited to, thermocouples, resistance temperature detectors, thermistors, and semiconductor based integrated circuits.

As used herein, the term “thermal dose profile” refers to prescribed or predetermined desired temperature change and a desired heating time period in order to achieve a selective neural conduction block, and an energy amount to be delivered from the controller device to the neural prosthetic device to achieve the desired temperature change for the time. The temperature change can determine the strength and size selectivity of the neural block. As thermal environment can be varying, a given temperature change may require a different amount of energy that can be altered with feedback-based control. The thermal dose profile can include safety and efficacy boundaries for amounts of heat that can be applied for given heating time periods.

As used herein, the term “inhibition” refers to a partial or complete stoppage of conduction through at least a portion of a nerve. Inhibition can be due to a reversible nerve block or an ablation.

As used herein, the term “patient” refers to any warm-blooded organism, including, but not limited to, a human being, a pig, a rat, a mouse, a dog, a cat, a goat, a sheep, a horse, a monkey, an ape, a rabbit, a cow, etc. The terms patient and subject can be used interchangeably.

### II. Overview

After damage from injuries or other pathological processes, small-diameter nerve fibers may conduct hyperactively or with hypersensitivity. Current treatments, such as pharmaceutical, surgical, and electrical neuromodulation treatments can have severe side effects and/or limited efficacy blocking conduction in small-diameter nerve fibers (also referred to as small-diameter axons). Traditional electrode-based neuromodulation modalities, such as direct current (DC) or high-frequency alternative current (HFAC), preferentially block large-diameter efferent axons because the transmembrane potential evoked by the extracellular electrode is proportional to the axon diameter. Therefore, additional efforts have to be made to selectively block the small-diameter axons, such as changing the stimulation frequency of HFAC. In addition, the selectivity gets further degraded due to electrode corrosion and dislocation during chronic implantation.

An alternative to traditional therapies is the application of heat to selectively inhibit small-diameter sensory fibers for the treatment of sensory disorders, like neuropathic pain. One example of heat application, infrared (IR) neural inhibition (INI), which relies on a laser-induced baseline temperature elevation that causes heat-induced acceleration of K₊ channel kinetics to block neural conduction, can selectively inhibit small-diameter slow-conducting nerve fibers. In addition to size selectivity, IR light can also achieve spatial selectivity. However, implementation of INI, or infrared stimulation more generally, in a living subject is difficult. For example, INI is not energy efficient, can require excess heating that can cause tissue damage to reach certain block thresholds, and has several implantation issues. Accordingly, described herein is a selective heat block treatment that can apply controlled heating (e.g., using a resistive heating element, an ultrasound heating element, a radio-frequency heating element, or the like) to achieve selective neuromodulation of small-diameter fibers. The system can deliver heat effectively to a selected one or more fibers in one or more specific neural sites in a patient's body while avoiding excessing heating of the one or more specific neural sites in a patient's body and surrounding tissues with a closed loop control of the thermal dose being applied. Additionally, the nerve interface of the system can avoid causing significant mechanical damage to the one or more specific neural sites in a patient's body and withstand mechanical disruptions from adjacent muscle movements. Finally, the system can be energy efficient, minimizing the need for recharging.

### III. Systems

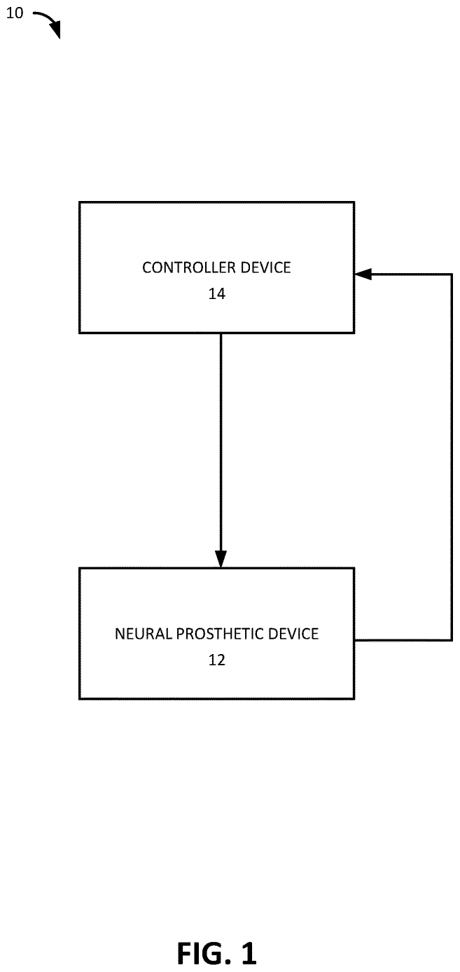

An aspect of the present disclosure can include a system 10, as shown in FIG. 1, that can apply controlled heating to achieve selective inhibition (e.g., by inducing a reversible heat-induced selective block and/or by selective heat ablation). The system 10 can include a controller device 14 and one or more neural prosthetic devices (shown as neural prosthetic device 12, which will be described singularly, but it should be understood that multiple devices may exist). The neural prosthetic device 12 and the controller device 14 can engage in closed-loop control that can affect the thermal dose delivered by the neural prosthetic device 12. The neural prosthetic device 12 can interface with one or more nerves to deliver heat for selective inhibition while avoiding excessive heating. The system 10 can employ one or more heating modalities (e.g., using a resistive heating element, an ultrasound heating element, a radio-frequency heating element, or the like) to deliver a selective heat block to nerve fibers. The one or more heating modalities described herein are superior to traditional heat-based block because they are able to be used in implantable form since the new one or more heating modalities are energy efficient, do not require excess heating that can cause tissue damage to reach certain block thresholds, and has reduce the likelihood of implantation issues.

The system 10 can apply controlled heating using, for example, heat sources, such as a resistive heating element, an ultrasound heating element, a radio-frequency heating element, or the like. The system 10 can achieve the selective inhibition, for example, by preferentially inhibiting small-diameter axons, such as sensory axons, which is unique compared to traditional inhibition modalities (e.g., pharmaceutical, electrical or the like) that preferentially inhibit large-diameter axons and can only inhibit small-diameter axons if the large-diameter axons are already inhibited. In addition to selectivity, the system 10 is energy efficient, giving the system 10 advantage when implanted in a subject's body to deliver the selective inhibition.

As shown in FIG. 1, the system 10 can include a neural prosthetic device 12 that can be positioned near and/or on a nerve (e.g., a peripheral nerve) and a controller device 14 that can deliver an amount of energy to the neural prosthetic device and automatically control the amount of heat applied via the neural prosthetic device. The neural prosthetic device 12 can be implanted around and/or on at least a portion of a nerve and selectively apply heat, based on the energy received from the controller device 14, to one or more locations on the nerve. As an example, the neural prosthetic device 12 can be a nerve cuff. The controller device 14 may be fully or partially implanted or entirely external and attached to the skin of the subject. The neural prosthetic device 12 and the controller device 14 can be in wired and/or wireless communication. The controller device 14 can deliver at least a predefined amount of energy to the neural prosthetic device 12 and can receive at least data from one or more portions of the neural prosthetic device (operating as a feedback loop).

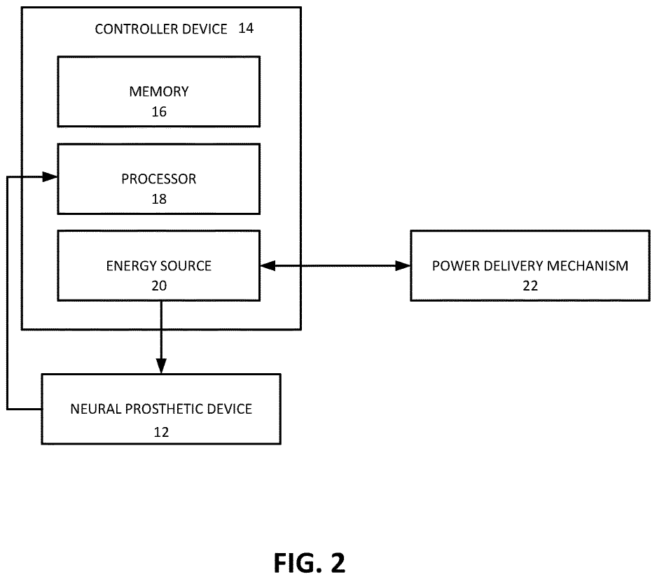

FIG. 2 shows the controller device 14 that can deliver an amount of energy to the neural prosthetic device 12 in greater detail. The controller device 14 can include, but is not limited to, a non-transitory memory (memory 16), a processor 18, and an energy source 20. The memory 16 can store instructions related to actions undertaken by the controller device 14 and these instructions can be executed by the processor 18 to cause the controller device 14 to execute one or more of the actions. The memory 16 can also store data related to delivery of energy to the patient (e.g., one or more parameters taken regarding the patient, one or more initial dose settings/requirements for one or more nerves, one or more thermal dose profiles for a patient using the system, and/or information regarding one or more determined heat boundaries for the one or more nerves). In some instances, the memory 16 and the processor 18 can be embodied together as a microprocessor or the like. The instructions and/or data stored in the memory 16 can be accessed and executed by the processor 18 to operate the energy source 20 to deliver a predefined amount of energy to the neural prosthetic device 12.

The energy source 20 (or any other part of the controller device 14) can receive power/energy from a power delivery mechanism 22. The power delivery mechanism 22 can connect at least a portion of the controller device 14 and an external power source (not shown, but examples include power from a wall outlet, power from a battery, etc.) and can deliver power from the external power source to the controller device 14. Based on the data stored in the memory 16, the controller device 14 can then send a predetermined amount of energy to the neural prosthetic device 12 where the amount of energy is converted to an amount of heat that can be applied to at least one portion of a nerve. The predetermined amount of energy can be an initial dose based on determined based on the initial dose settings/requirements for the nerve being treated. The predetermined energy can, additionally or alternatively, be based on one or more of the thermal dose profiles for the patient and/or the predetermined heat boundaries for the patient (e.g., for the initial dose and/or for an updated dose based on feedback received from the neural prosthetic device 12). For example, the feedback received from the neural prosthetic device 12 can be based on a monitored temperature of the nerve; the predetermined amount of energy can be altered to maintain the temperature of the nerve within the thermal dose protocol of the patient and the predetermined heat boundary for the nerve.

The thermal dose profile can illustrate the overall heat and time needed to cause inhibition of a certain nerve fiber (or class of nerve fibers). In some instances, the thermal dose profile can include a limit for how hot the elements can get for an amount of time to reach the overall heat as a heat boundary that ensures safety (e.g., a temperature of 40 degrees Celsius is unsafe for the elements for 5 minutes, but 40 degrees Celsius may be permissible for 10 seconds). The thermal dose profile may be specific for a patient and/or a nerve and/or fiber (e.g., different sized nerve fibers may have different dose profiles). The heat boundaries can be based on group research data, theoretical data, patient specific data or a combination thereof. The heat boundary may be changeable by a medical professional.

The thermal dose profile can include a temperature elevation or change corresponding to a certain heating period required for that desired temperature elevation or change. The temperature elevation or change can determine strength and/or size selectivity of block of a set of one or more fibers in the nerve. The information regarding a determined heat boundary for the nerve can include, for example, one or more temperature safety boundaries for repairable damage to one or more parts of the nerve (and/or nerve fiber) and/or for irreparable damage to one or more parts of the nerve (and/or nerve fiber).

The power delivery mechanism 22 can deliver power from an external power source (e.g., a battery, a power outlet, or the like) to the energy source 20 of the controller device 14 (or any other component of the controller device 14). The energy source 20 can store an amount of energy needed to run the system 10 for a given amount of time (e.g., 1 hours, 5 hours, 1 day, 2 days, etc.) before needing to be recharged by the power delivery mechanism. The controller device 14 may notify the patient that the energy source 20 needs recharged (e.g., by an audible alert, a haptic alert, a visual alert, or the like). The notification may be made by the system itself or an external mobile device (not shown) in wireless communication (e.g., WIFI or BLUETOOTH) with the system.

The controller device 14 can also include a safety mechanism and protocol that can include a failure detection mechanism (and/or module stored in memory 16) (not shown). The failure detection mechanism can detect a fault (e.g., an electrical or mechanical fault) within any part of the system 10. A fault can occur if an integrity of at least a portion of the neural prosthetic device being compromised, for example, causing an internal electric circuit to be exposed to the patient. The controller device 14 can cut power to at least the heat delivery component 24 of the neural prosthetic device when the failure detection mechanism detects the fault.

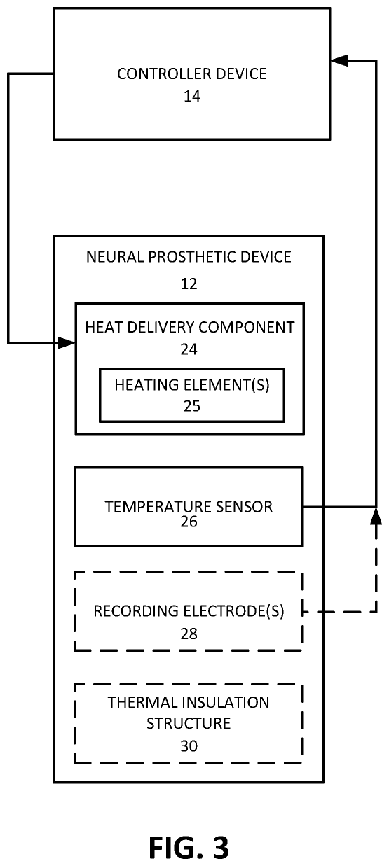

FIG. 3 shows the neural prosthetic device 12 in greater detail. As an example, the neural prosthetic device 12 can be at least partially implanted in a subject's body. The neural prosthetic device 12 can communicate with the controller device 14 in a bidirectional manner. In one direction, the controller 12 can send energy to the neural prosthetic device 12 that can convert the energy to a heat signal that is delivered to one or more nerve fibers. The neural prosthetic device 12 can include a heat delivery component 24 configured to convert the amount of energy received from the controller device 14 to an amount of heat and to deliver the amount of heat to at least one part of the nerve to block conduction in at least a portion of the nerve or nerve fiber. Based on the delivery of the heat signal, the neural prosthetic device 12 can send feedback to the controller device 14, and the controller device 14 can use the feedback to configure the energy that is delivered to the neural prosthetic device. The neural prosthetic device 12 can also include at least one temperature sensor 26 that can record and/or monitor a temperature of at least one part of the nerve or the nerve fiber, and the temperature can be sent back to the controller device 14 as feedback that may be used to recalculate the energy delivered to the heat delivery component 24. The neural prosthetic device 12 can have a high enough energy efficiency so that at least a portion of the neural prosthetic device 12 can have an implantable design. For example, the neural prosthetic device 12 muse operate for a period of time without recharging. High energy efficiency can increase battery longevity and permit more functionalities like closed loop control.

The neural prosthetic device 12 may also include one or more recording electrode (recording electrode(s) 28) that can detect neural signals indicative of pain in one or more portions of the nerve and/or a thermal insulation structure 30 that can surround at least a portion of the heat delivery component 24 to reduce heat dissipation. When the neural prosthetic device 12 includes one or more recording electrodes (recording electrode(s) 28) then the controller device 14 in communication with the neural prosthetic device can receive the neural signals indicative of pain from the one or more recording electrode. The controller device 14 can determine the patient's level of pain based on the received neural signals and can alter the amount of energy sent to the heat delivery component 24 of the neural prosthetic device 12 based on the patient's level of pain to better treat the pain. For example, the amount of energy can be increased iteratively, within the thermal dose profile and predetermined heat boundary, until the neural signals indicate a lessening and/or stoppage of pain and then that amount of energy can be maintained for a period of time.

The heat delivery component 24 of the neural prosthetic device can include at least one heating element (heating element(s) 25). The at least one heating element (heating element(s) 25) can be dispersed throughout the neural prosthetic device 12 around the circumference and/or along the length of the nerve for controlled and specific positioning of heat on at least one portion of the nerve. The at least one heating element (heating element(s) 25) can include at least one resistive heating element, ultrasound heating element, or radio-frequency heating element. The at least one heating element(s) can efficiently deliver heat to one or more spatial locations on the nerve to preferentially select small-diameter nerve fibers for inhibition (e.g., with little or no inhibition or stimulation of large-diameter nerve fibers). For example, pain can be treated with heat delivery from the system herein without causing changes (or significant changes) in conduction of nerves related to motor function. In some instances, the heat delivery component 24 can include at least a plurality of heating elements. The controller device 14 can select one or more of the plurality of heating elements (heating element(s) 25) to control a spatial location on the nerve where the amount of heat is delivered.

The one or more temperature sensor (temperature sensor 26) can be positioned within the neural prosthetic device 12 at one or more circumferential and/or longitudinal locations along the nerve to record temperature of the nerve. The recorded nerve temperature(s) can be received by the controller device 14. The controller device 14 can determine if the amount of energy delivered to the neural prosthetic device 12 is too high or too low based on the recorded nerve temperature(s) and adjust the amount of energy accordingly (e.g., to be within the heat boundary and/or to be closer to a desired temperature of the thermal dose profile). The adjustment can be closed loop control such as, but not limited to, one or more of PID control.

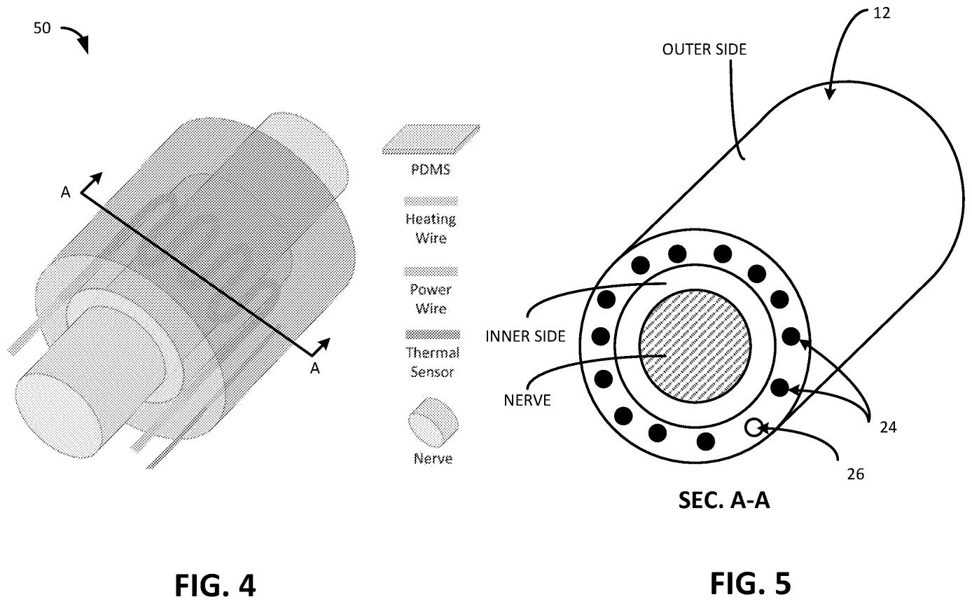

FIG. 4 shows an exemplary illustration 50 of a neural prosthetic device as a durable, biocompatible nerve cuff (e.g., shaped as a circle, but may be shaped as an oval, square, spiral, etc.) that uses at least one heating element (shown as a resistive heating element, but may also be an ultrasound heating element, a radio-frequency heating element, or the like) to convert electricity into heat. While nerve cuff of FIG. 4 can be used for ex vivo or acute in vivo experiments/uses, an important feature is the neural prosthetic device can be implantable. Importantly, the nerve cuff can deliver a heat-induced selective block (e.g., selective for small-diameter fibers) using resistive heating or other types of non-IR heating. Accordingly, the nerve cuff can include other heating elements embedded into the cuff as long as such heating elements meet the standard of durability and biocompatibility (e.g., only one heating wire element is shown in FIG. 4, but the nerve cuff can include multiple independent heating elements). For example, the multiple independent heating elements can be located on different sides of the nerve cuff to allow for a spatial selective application of the heat, which can be a useful feature for large nerves with several fascicles. Additionally, the nerve cuff is designed so that no bare metal is exposed, and no electrochemical reactions are relied on to generate heat.

At least the nerve interface of the neural prosthetic device should avoid inducing significant mechanical damage to the nerve, so at least the nerve interface can be soft and not constraining the nerve from swelling post-implantation. Also, since most peripheral nerves are located right next to muscles, the at least the nerve interface can stand the mechanical disruptions (e.g., tearing, rubbing, etc.) during normal muscle movements. As shown in FIG. 4, the heating element can include an embedded heating wire element for inducing heat into the nerve.

As shown in FIG. 4 the nerve cuff can include an inner side that can face the nerve (the inner side can contact one or more portions of the nerve or merely surround the nerve) and an outer side can face away from the nerve. At least the heat delivery component (shown as heating wire that may be double insulated by being coated with biocompatible materials and embedded in a biocompatible silicon polymer) and the temperature sensor (shown as thermal sensor) can be sandwiched between the inner and outer sides. A power wire can extend from the heat deliver component to the controller device (not shown). The inner and outer sides can be fabricated of a polymeric organosilicon compound, such as polydimethylsiloxane (PDMS) for example, and can have varying thicknesses. The temperature sensor (thermal sensor) can be, for example, a thermocouple positioned on or near the inner side of the heat deliver component facing the nerve. The nerve cuff can be attached around the nerve via one or more common surgical methods (e.g., sutures, biocompatible glue, etc.). FIG. 5 shows a cut section A-A of the exemplary neural prosthetic device 12 of FIG. 4 to show the inside of the nerve cuff. The heating delivery component 24 is shown with multiple axial locations around the nerve (and can be any number one or more at any given location longitudinally along the nerve depending on the pattern in which the heating wire is bent). Only one temperature sensor 25 is shown, but it should be understood that many different temperature sensors may be included.

For example, the exemplary neural prosthetic device as shown in FIGS. 4 and 5 can be used for treating pain of a patient with heat based selective inhibition. The neural prosthetic device and controller device (not shown in FIGS. 4 and 5) can be at least partially implanted in the patient with the neural prosthetic device positioned around at least a portion of a nerve known to include at least one sensory fiber. One or more thermal dose protocols for the treatment of pain can be loaded into and/or stored in a portion of the controller device. A medical professional may determine the appropriate thermal dose protocol(s) for the patient. A thermal dose protocol can include the amount of heat and duration of the heat necessary to cause an inhibition in small-diameter fibers of the nerve for the block of sensory (pain signals) to the patient. The thermal dose protocol can also include the amount of energy and different heating combination required to generate the amount of heat for the duration and spatial selectivity information regarding which portion(s) of the nerve should be heated for the desired inhibition.

The medical professional and/or the patient can manually activate the system so that the controller device can begin sending energy to the neural prosthetic device according to the thermal dose protocol. The heat delivery component of the neural prosthetic device can include one or more heating elements (heating wire in FIGS. 4 and 5) that can convert the energy sent from the controller device (e.g., electrical energy) into heat energy. The heat energy can then be applied to one or more portions of the nerve via the one or more heating elements based on the thermal dose protocol (e.g., heat can radiate from the heating wire in one or more directions). The neural prosthetic device can more efficiently and accurately heat one or more portions of a nerve as compared to infrared optical heating systems. After the neural prosthetic device has begun the provide heat to the nerve one or more temperature sensors of the device can measure and record the temperature of at least one portion of the nerve. The controller device can receive one or more signals including the recorded temperature(s) and alter the energy provided to the neural prosthetic based on the recorded temperature(s) (e.g., increase energy or maintain energy for a longer time period if the temperature is too low, decrease energy if the temperature is too high, or the like). One or more recording electrodes positioned near and/or on the nerve (either as part of the neural prosthetic device or separate entities) can record neural signals indicative of, in this example, pain. The recorded neural signals can be communicated to the controller device (via wired or wireless communication). The controller device can determine based on the recorded neural signals if the selective inhibition is working (e.g., if the patient is feeling any pain or pain above a predetermined threshold) and/or if one or more portions of the therapeutic dose profile and/or the amount of energy sent to the neural prosthetic device should be altered. The controller device can then make the alterations (within the heat boundaries) and complete the closed loop control to effectively treat the patient's pain. In some instances, the patient may also be able to control the start and stop and increase of the selective inhibition (within safety and efficacy bounds) via an external device in communication with the controller device.

### IV. Methods

Another aspect of the present disclosure can include method 60 for a treatment that can apply controlled heating to achieve selective inhibition of small-diameter fibers. The method 60 can be executed using the systems 10 and 50 as shown in FIGS. 1-5 as well as other components of systems 10 and 50 described herein but not pictured. For purposes of simplicity, the method 60 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method 60, nor is method 60 limited to the illustrated aspects.

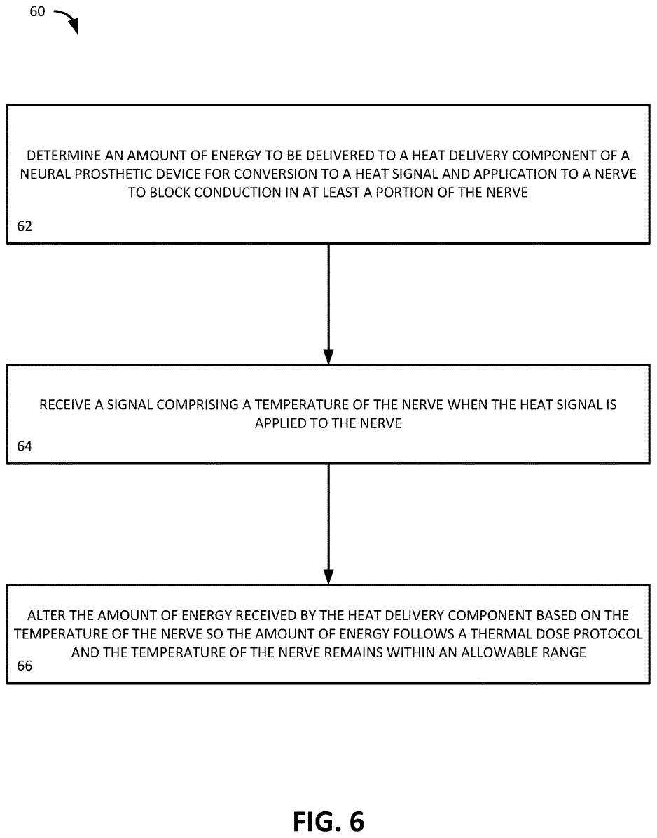

Referring now to FIG. 6 illustrated is a method 60 for applying controlled heat to achieve selective inhibition of small-diameter fibers. Conduction can be blocked in at least a portion of the nerve as a treatment for pain, without causing loss (or significant loss) of motor function. At 62, an amount of energy to be delivered to a heat delivery component of a neural prosthetic device for conversion to a heat signal and application to a nerve to block conduction in at least a portion of the nerve can be determined. The amount of energy to be delivered by the controller can be determined based on a thermal dose profile and/or a heat boundary, which can be stored by a controller. The controller can also receive at least one input selecting at least one fiber in the nerve for treatment. For example, the controller can select the small-diameter nerve fiber(s) that are more sensitive to heat (size selection) or select at least two fibers in a region near each other (spatial selection). At 64, a signal including a temperature of the nerve when the heat signal is applied to the nerve can be received by the controller. The signal can be recorded and sent to the controller by a temperature measurement component (such as a temperature sensor like a thermocouple. The controller can also receive another signal including an indication of pain that can be sent from one or more electrophysiological recording electrodes that can detect pain signals from the nerve. At 66, the amount of energy received by the heat delivery component can be altered based on the temperature of the nerve, so the amount of energy follows the thermal dose protocol, and the temperature of the nerve remains within an allowable range. Additionally and/or alternatively, the selected one or more axons to be inhibited with the heat signal can also be altered based on the temperature change (e.g., the temperature recorded by the temperature measurement component). For example, a different one or more axons can be inhibited with the altered heat signal.

As an example, the neural prosthetic device can be a cuff that can be fabricated by:
1. Layering down a silicone polymer on a glass substrate;
2. Putting a folded/coiled heating wire element on top of the first silicone polymer layer;
3. Layering down another silicone polymer layer on top of the heating element to embed the heating element;
4. Peeling the product from step 3 from the substrate, stretching the product from step 3, and laying the stretched product from step 3 back on to the glass substrate;
5. Applying another thin layer of silicone polymer on top of the stretched silicone polymer to induce self-warping after removing from the glass substrate.

Other methods not shown are contemplated. For example, the controller can also include at least one safety measure such as for fault detection. The controller can detect occurrence of a fault (e.g., with a fault detection mechanism and/or module). The fault can include the integrity of at least a portion of the neural prosthetic device being compromised, causing an internal electric circuit to be exposed or an electrical fault. Additionally, the controller can include a rechargeable energy source from which the energy that is transformed to heat is drawn. The controller can receive power from an external source (such as a battery or power outlet according to a wired or wireless connection). The controller can then deliver at least a portion of the power to the neural prosthetic device.

### V. Experimental

The following experiment shows that selective inhibition can be achieved under different heating modalities (infrared neural inhibition (INI) and resistive heating) and that resistive heating can be used to replace INI. Resistive heating is attractive because it can be designed to minimize spatial variance of induced temperature elevation, can be applied using a relatively simple apparatus, and can provide better energy efficiency compared to INI, each of which may permit the development of battery-powered implantable devices, increasing convenience of implementation on small-diameter axons for basic research and translational applications.

### Methods

### Animal Model

The hypothesis was tested in vitro (N=6) on the pleural-abdominal connective nerve of Aplysia californica (278±40 g, South Coast Bio-Marine, CA). The nerve consists only of unmyelinated axons with large and small diameters, because Aplysia does not make myelin. This provides a robust testing platform for modifying neural activity based on axonal size differences without having to consider the effects of myelination. It has been shown that results obtained in Aplysia can readily be translated to vertebrate systems as Aplysia provides a robust model for unmyelinated, pain-conducting C-fibers in vertebrates.

### Electrophysiological Recording

Customized suction electrodes (0.35 mm inner diameter) were 3D-printed with a Form 3 3D printer (Formlabs, MA, USA) and used to electrically stimulate and record CAPs at 2 Hz with 2 ms symmetric biphasic current pulses (1 ms per phase). The current amplitude was adjusted between 0.3-0.5 mA to ensure full recruitment of all CAP components. The pulse signal was generated using a pulse stimulator (Model 2100, A-M Systems, WA, USA) and converted into current pulses using a stimulus isolator (A395, World Precision Instruments, FL, USA). The evoked CAPs were amplified and filtered (×10,000, 100-500 Hz) using a differential AC amplifier (Model 1700, A-M Systems, WA) and then digitized with a data acquisition (DAQ) device (USB-6003, National Instruments, TX, USA) using AxoGraph X software (AxoGraph, CA, USA). Different axon subpopulations (large vs. small) were identified based on the differences in the latencies of the corresponding compound action potential (CAP) components. Axonal subpopulations with different diameters have different conduction velocities (i.e., larger diameters correspond to faster conduction velocities), which permits the analysis of responses from different nerve subpopulations. During the experiments, the nerve was placed in a chamber filled with Aplysia saline (460 mM NaCl, 10 mM KCl, 10 mM MOPS, 10 mM glucose, 22 mM MgCl2·6H2O, 33 mM MgSO4·7H2O, 13 mM CaCl2, pH 7.5) at room temperature (˜22° C.) to sustain the health of the nerve during the tests.

### Resistive Heating

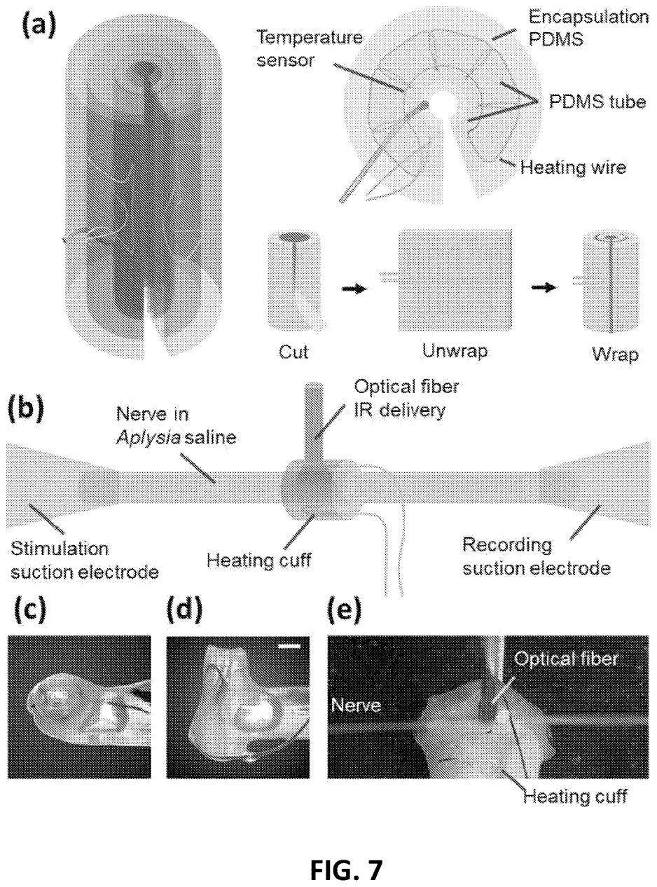

FIG. 7, element a illustrates the fabrication steps of the miniature resistive heating cuff (the heating cuff in FIG. 1, element b): 1) Cut the outer PDMS tube (1.575 mm inner diameter, McMaster-Carr, OH, USA) axially and unwrap it into a flattened sheet; 2) sew the nichrome heating wire (#761500, 25.4 μm bare diameter, A-M Systems, WA, USA) into the unwrapped outer PDMS tube; 3) re-wrap the outer PDMS tube around the inner PDMS tube (1.016 mm inner diameter, McMaster-Carr, OH, USA); 4) sew the thermocouple (5SC-TT-T-40-36, dia.=200 μm, Omega Engineering, CT, USA) through both layers of the PDMS tube and place it on the inner surface of the cuff; 5) cut the inner cuff along the slit of the outer PDMS tube; 6) encapsulate the entire heating cuff assembly with silicone adhesive (KWIK-SIL, World Precision Instruments, FL, USA) while using a steel pin (0.024 in. diameter, McMaster-Carr, OH, USA) as the place holder for the nerve; 7) cut a slit throughout all layers of material along the existing cut of the outer PDMS tube; 8) remove the pin to leave an opening for the nerve after curing the silicone adhesive; and 9) trim excessive silicone adhesive. FIG. 7, element b shows an example experimental system that includes the heating cuff and an optical fiber for delivery of infrared light for comparison purposes.

The constructed heating cuff is shown in FIG. 7, elements c, d, and e. The heating cuff was applied by sliding a nerve into the core channel of the heating cuff via the slit. There was a 4.5 mm long region of the core channel that was surrounded by the embedded heating wire. Two multi-strand copper wires (30 American-wire-gauge) were used to connect the heating cuff to the temperature controller. The total DC resistance (including the connection wires) was 40Ω, including a 3Ω resistance of the contacts and a 37Ω resistance of the heating wire embedded in the cuff. A modified temperature controller (TC-324C, Warner Instruments, MA, USA) was used to control the DC current level passing through the heating wire.

### Infrared Neural Inhibition (INI)

INI was conducted by delivering IR light to the targeted nerve region via an optical fiber (P600-VIS-NIR, Ocean Insight FL, USA), which had a 600 μm core diameter with an NA=0.39. A single-mode laser diode (λ=1470 nm, QFBGLD-1470-250, QPhotonics, MI, USA) and a controller (6340-4A, Arroyo Instruments, CA, USA) were used to generate IR light at the desired power level. The laser diode temperature was held constant at 20° C. to maintain stable optical power output. A DAQ device (USB-6218, National Instruments, TX, USA) was used to trigger the 60-second laser pulse train (1250 Hz, 400 us pulse width). The relationship between the laser diode current and actual IR optical power at the fiber tip was measured using a power meter (PS19Q, Coherent, CA, USA) to guide the execution of the experiment.

### Temperature Elevation Measurement

Resistive heating and INI were applied to the same nerve segments. The temperature elevation (ΔT) during heating was measured using a type-T thermocouple (5SC-TT-T-40-36, dia.=200 μm, Omega Engineering, CT, USA). The thermocouple was positioned near the nerve surface as shown in FIG. 7, element b. A thermocouple-to-analog converter (SMCJ-T, Omega Engineering, CT, USA) was used to convert the signal from the thermocouple to an analog voltage signal (0-100 mV for 0-100° C.), which then was digitized and recorded using the same DAQ device as for the CAPs. ΔT was calculated as the temperature during the heating period minus the baseline temperature during the control period before heating. The saline level in the chamber was controlled to ensure full immersion of the heating cuff, IR delivery fiber tip, and nerve to provide a stable thermal environment.

### Experimental Protocol

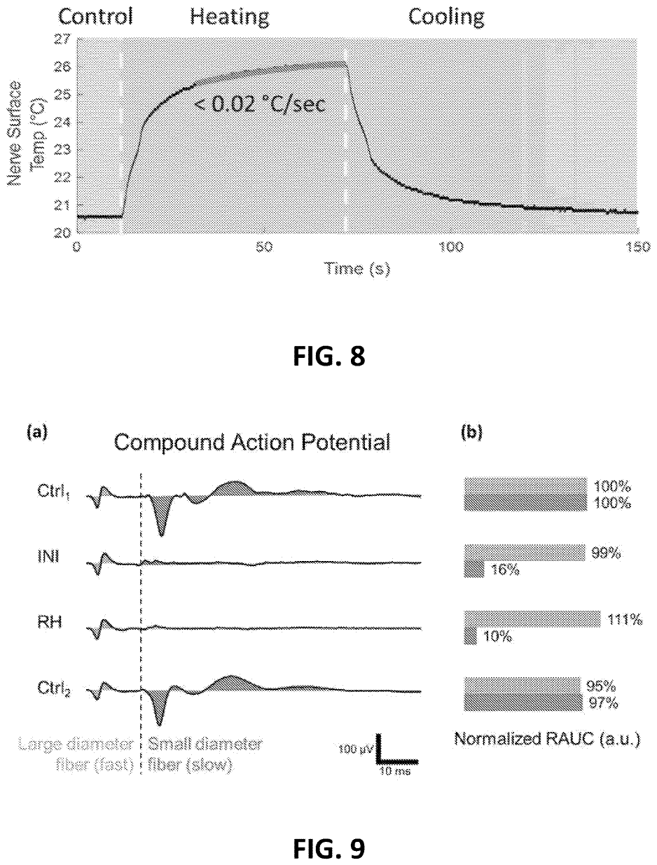

The experiments were performed in excised pleural-abdominal connectives from Aplysia to test the effects of INI and resistive heating on neural conduction. Six nerves were tested. Each nerve was exposed to both INI and resistive heating. Three nerves were first tested with INI, and the other three were first tested with resistive heating. Each heating modality was tested by conducting a series of 150-second heating trials. Electrical stimulation was conducted for the duration of each heating trial to assess the nerve response and determine whether the response induced by the current heating modality met the defined endpoint. Each 150-second heating trial consisted of a 10-second control period (no heating), a 60-second heating period, and an 80-second cooling period, as shown in FIG. 8. The heating trials were repeated with increasing power applied at the neural interface until at least partial inhibition was evident, or the maximum ΔT was reached, at 15° C. This 15° C. ΔT limit was derived from our previous experience testing INI with Aplysia (data not shown). The maximum ΔT value increased by approximately 2° C. for each repetition of the heating trial. The inhibitory effect was assessed by comparing the CAPs during the initial 10-second control period and the 60-second heating period. The acute health conditions of the nerves after heating were assessed by comparing the CAPs during the initial 10-second control period and the 80-second cooling period.

### Data Analysis

### CAP Recording Pre-Processing

To prepare the data for analysis, three pre-processing steps were applied: selecting the CAPs during stable ΔT, segmenting the CAP into subcomponents that correspond to large- and small-diameter axons, and subtracting the contribution of noise. A description of the detailed processes follows.

To compare the inhibitory effect in response to different ΔT values, only the CAPs during the stable ΔT were included in the analysis. As shown in FIG. 8, when heating was applied, the temperature increased rapidly during the initial 15 s and then stabilized. The quasi-steady temperature region was empirically defined as corresponding to a temperature change rate<0.02° C./s (as shown by the thicker curve of a different color in the heating portions of FIG. 8). Only CAPs during this quasi-steady condition were included in the analysis. Preliminary tests indicated that the inhibition effect was not consistent during rapid temperature changes. It should be noted that the dashed lines indicate the time point between different phases of the heating trial. The different colored curve indicates where the temperature was considered quasi-stable (changing rate<0.02° C./sec) and used for ΔT calculation and data analysis.

To quantify the inhibitory effect on the large- and small-diameter axonal subpopulation, the CAPs were segmented into fast- and slow-conducting subcomponents. Because all axons were stimulated simultaneously by the suction electrode, the latency of the CAP subcomponents arriving at the recording electrode was inversely proportional to the conduction velocity. For unmyelinated axons, large-diameter axons usually have a higher conduction velocity and are responsible for motor output, whereas smaller axons have a slower conduction velocity and are responsible for sensory input.

The segmentation of CAPs was done by defining the two outer boundaries that cover the whole length of the CAP and an inner boundary between the fast- and slow-conducting subpopulations. Although the general CAP shape and conduction velocity distribution remain similar across nerves, the conduction velocity can shift over time owing to fatigue, temperature, or variance across preparations. As a consequence, the segmentation boundaries of CAPs were detected by a custom algorithm and manually inspected for each nerve. The outer boundaries of the CAPs were detected as the CAP signal amplitude rises/falls beyond the normal range of background noise (mean+3*standard deviation). The inner boundary between the fast- and slow-conducting subpopulations was selected by determining the minimum value for the variance across heating trials. This minimized the fluctuation caused by the shift of CAP subcomponents across the inner boundary. The boundary detection results were inspected to determine that the boundary did not bisect CAP components, and in 97/111 trials, no adjustments were necessary.

Two signal processing steps were conducted to minimize the contribution of noise to the subsequent analysis: 1) the DC components from the recorded CAPs were removed to avoid any drift due to electrodes or circuitry. 2) For each heating trial, a short recording period from the CAP channel was recorded when electrical stimulation was not applied. The background noise sample and its properties were then used to subtract the noise contribution from subsequent analyses.

### Quantification of Inhibition Effect

To quantify inhibition strength, the rectified area under the curve (RAUC) for the fast- and slow-conducting subpopulations in the CAP, as shown in FIG. 9, elements a and b, were independently calculated. The RAUC during the heating period was normalized to the average RAUC during the last three seconds of the control period. The normalized inhibition strength (NIS) can then be calculated as the reduction in the normalized RAUC:

Normalized inhibition strength=1−Normalized RAUC.

The normalized inhibition strength will increase from 0 up to 1 when an inhibitory effect is present. A normalized inhibition strength below 0 indicates an excitatory effect. It can be compared across different subpopulations and nerves as it does not depend on the absolute value of the RAUC.

Inhibition probability was calculated as the number of inhibition events divided by the total number of CAP recordings. A CAP recording was considered an inhibition event when its normalized inhibition strength was greater than 50%. To quantify the change in the inhibition probability as ΔT increased, the normalized inhibition strength data from all nerves were pooled and grouped into non-overlapping 1° C. ranges (e.g., 0-0.99° C. and 1-1.99° C.) based on their corresponding ΔT. The inhibition probability was calculated for each 1° C. range using normalized inhibition strength data within that range. This inhibition probability was calculated separately for fast- and slow-conducting subpopulations.

To compare thresholds of inhibition between INI and resistive heating, probit regression was applied to the inhibition probability data. Probit regression is suitable for assessing responses from experiments with binominal results. Previous studies on laser-tissue interactions have applied probit regression to characterize the response during laser ablation and infrared stimulation. For the probit regression, a normal cumulative distribution function (CDF) was fitted to the inhibition probability in response to an increase in ΔT. The probit regression function is:

$$Fitted inhibition probability ( p ) = \frac{1}{2} [ 1 + erf ( \frac{Δ T - {T}_{50}}{δ \cdot \sqrt{2}} ) ] ,$$

where ΔT is the independent variable. The fitting process results in an estimate of the T₅₀ for inhibition, which is the temperature elevation threshold for a 50% probability of inhibition. It also estimates δ, which is the standard deviation of ΔT. Probit regression was conducted separately for the slow-conducting subpopulation during the INI and resistive heating tests. The T₅₀ parameters of the fitted models were compared to determine the temperature elevation threshold difference between the two heating methods.

To characterize the selectivity of inhibition in the slow-conducting subpopulation, the inhibition selectivity index was calculated as:

$$Inhibition selectivity index = \frac{NIS of slow}{NIS of slow + NIS of fast} ,$$

where “NIS of slow” means the normalized inhibition strength of the slow-conducting subpopulation and “NIS of fast” means the normalized inhibition strength of the fast-conducting subpopulation. The inhibition selectivity index was only calculated for CAPs in which a normalized inhibition strength greater than 50% was observed for at least one subpopulation, across the whole temperature range. An inhibition selectivity index between 0.5 to 1 indicates a selective inhibition of the slow-conducting subpopulation. An index of 1 indicates an inhibition effect only on the slow-conducting subpopulation. On the opposite, an inhibition selectivity index between 0 to 0.5 indicates a selective inhibition of the fast-conducting subpopulation. An index of 0 indicates an inhibition effect only on the fast-conducting subpopulation. A paired t-test of the inhibition selectivity index was conducted to compare whether size-selectivity was statistically different between the two heating modalities.

### Characterization of the Heating Process and Thermal Dose

While it is important to explore the electrophysiological response for both heating modalities, the heating process is also important for the potential implantable design with those heating modalities. Here the efficiency of the power applied at the neural interface and the efficiency of total electrical power were characterized separately, to have a comprehensive understanding of the heating process.

The efficiency of the power applied at the neural interface can be evaluated as the final ΔT (the average ΔT during the last three seconds of each heating trial) when a given power was applied at the neural interface. This parameter indicates how effective the modality is for coupling power into the tissue for generating ΔT. For INI, the power applied at the neural interface is the optical power of IR light emitted from the optical fiber tip. For resistive heating, the power applied at the neural interface is the electrical power delivered to the heating wire. Since two distinct heating processes exist here (volumetric energy deposition by INI and surface heat conduction by RH), it is necessary to evaluate the difference in the efficiency of the power applied at the neural interface between the two heating modalities. Higher efficiency of the power applied at the neural interface means less power will be dissipated into the surrounding tissue, which is potentially preferred from a safety perspective.

The efficiency of total electrical power can be calculated as the total electrical power (the electrical power consumed from the wall outlet) required for achieving a target final ΔT. Higher efficiency of total electrical power will permit a longer running time for a battery-powered implantable design. If the implant design is externally powered, a smaller electrical power requirement can also lower the difficulty of wireless power delivery. For INI, the total electrical power is calculated as the electrical power consumed by the laser diode. For resistive heating, the total electrical power is calculated as the power consumed by the entire heating circuit, including the cable connecting the heating wire to the temperature controller. The electrical power consumed by the controlling device (e.g., the temperature controller or laser diode driver) was not included in this study because it involved irrelevant power consumption such as the power for the display. In addition to heating efficacy and overall energy efficiency, it is also important to examine the repeatability of the heating response across different nerves.

To identify any potential methodological bias between the two heating modalities, it is necessary to compare the thermal dose applied to the nerve by each heating modality. However, calculating the commonly used cumulative temperature elevation dose (CEM₄₃) using absolute temperature is not applicable since Aplysia is a heterothermic animal. As Aplysia's natural habitat is intertidal pools, its body temperature can be changed by the environment, rather than maintaining a constant body temperature as is done by mammals. Therefore, the thermal dose for each heating modality was calculated as follows as the first approximation:

$$Thermal dose = \sum Δ T \cdot duration of each Δ T$$

### Simulation of the Temperature Elevation

The temperature elevation induced by the INI and resistive heating were simulated using COMSOL Multiphysics® (COMSOL) software and a mesh-based Monte Carlo simulation in the MATLAB® environment (MMClab) for light scattering and absorption. For INI and resistive heating, 3D models representing the experimental setup (e.g., the nerve and heating element) were recreated in Solidworks at the same dimension as in the real world. The nerve's diameter was approximated as 600 μm based on the measurement (614±70 μm). A saline object (10 mm×5 mm×10 mm) was created as the background object to bisect and surround all the other objects. The 3D objects representing each type of material were then imported into COMSOL for generating the corresponding tetrahedral mesh. The mesh density and quality were controlled to satisfy the requirement of heat transfer simulation and MMClab.

There are some common settings in the simulation for both INI and resistive heating: Most of the physical properties were assigned to each object domain using the built-in material in COMSOL: The saline domain was modeled using the water (liquid) material. The PDMS cuff was modeled using silicone material. The heating wire was modeled using the nichrome (solid, steady-state) material. The optical fiber was modeled using silica glass material. Some objects (e.g., nerve and heating wire) required the use of externally referenced physical properties, as shown in Table 1. The nerve's thermal property was approximated based on the nerve's water content. Laminar flow caused by the density change during heating was simulated in the laminar flow module for both INI and RH. Gravity was included in the laminar flow module as the negative direction along the z-axis. Heat dissipation caused by laminar flow was simulated using a COMSOL's multiphysics module: nonisothermal flow, The top surface of the saline was defined as the interface between ambient air and saline, where heat loss due to radiation was included in the simulation. All other outer surfaces of the saline were defined as symmetric boundaries where heat transfer and flow rate were both negligible. All objects and the ambient air were set to have an initial temperature of 22° C. The reference air pressure was 1 atm and the top surface of the saline object was defined as an open boundary with no stress in the laminar flow module. The model was simulated with a time-dependent solver for 0 to 60 s (the same heating period as in the heating protocol), with a 0.1 s output interval and physics-limited dynamic time steps. To validate the models, the temperature elevation distribution was simulated at the thermocouple location with several power levels applied at the neural interface. The simulated temperature elevation was then compared with the experimental result to validate the model. After validation, the required power to be applied at the neural interface for achieving the 50% probability of inhibition in the slow-conducting subpopulation was modeled, by each heating modality separately.

TABLE 1
Externally referenced physical properties of the materials

| Object | Property | Value | Unit |
|---|---|---|---|
| Heating | Thermal | 15 | W/(m · K) |
| Wire * | conductivity | | |
| | Heat capacity | 465 | J/(kg · K) |
| | at constant | | |
| | pressure | | |
| | Relative | 800 | a.u. |
| | permittivity | | |
| Nerve | Thermal | 0.49 | W/(m · K) |
| | conductivity | | |
| | Heat capacity | 3581 | J/(kg · K) |
| | at constant | | |
| | pressure | | |
| | Density | 1106 | kg/m³ |

* Directly from the supplier: A-M Systems, WA, USA

For resistive heating, the heat transfer in solids and fluids (in nerve, saline, PDMS, and heating wire), laminar flow (in saline), and electric currents (in heating wire) were simulated using existing COMSOL modules by those names. The heat source for the heater transfer was from the multiphysics module: electromagnetic heating. The power levels simulated for heating response validation was from 25 mW to 150 mW with a 25-mW step. A simulation of 103.1 mW power applied at the neural interface by resistive heating was performed to evaluate the temperature elevation across the nerve when a 50% probability of inhibition of the slow-conducting subpopulation was achieved experimentally.

For INI, the heat transfer in solids and fluids (in nerve, saline, and PDMS) and laminar flow (in saline) were simulated, using existing COMSOL modules by those names. The heat source for the heat transfer module was generated using MMClab. The LiveLink™ for MATLAB® was used to import the generated tetrahedral mesh from COMSOL as the mesh data for MMClab. Using the same mesh as the COMSOL model can help reduce the computational load when the MMClab result was imported back into COMSOL and mapped to the mesh nodes. In MMClab, optical properties for each type of material (nerve, saline, PDMS, and optical fiber) were assigned to the corresponding mesh element, as shown in Table 2. The nerve's optical properties were approximated using the average values from other species as there is no measurement available for Aplysia's nerve. The optical property of Aplysia saline was approximated by the values for water. A cone-shaped light source was used to mimic the light output profile from the optical fiber tip in an aqueous medium. Since the simulation's goal was to estimate the temperature distribution at the end of the heating period, the temporal fluctuations of temperature caused by each laser pulse were not investigated. Instead, the energy deposition of the laser pulse train using a continuous (CW) laser source with equivalent average optical power as the pulsed laser were simulated. The MMClab generated the normalized impulse response as energy deposition at each mesh node over time then converted to the step response for a unitary CW source by convolution, which means integrating over time and multiplying by the time step in the discrete domain. This energy deposition caused by the unitary CW source was then imported back into COMSOL. The unitary response by the optical power to be simulated was scaled and used as the heat source for the heat transfer in solids and fluids module. The power levels simulated for heating response validation was from 15 mW to 75 mW with a 15-mW step. A simulation of 52.4 mW power applied at the neural interface by INI was performed to evaluate the temperature elevation across the nerve when a 50% probability of inhibition of the slow-conducting subpopulation was achieved experimentally.

TABLE 2
Optical parameters of the materials

| | μₐ | μₛ | Anisotropy | Refractive |
|---|---|---|---|---|
| Material | (1/cm) | (1/cm) | (a.u.) | Index (a.u.) |
| Saline | 30.0 | 0 | N/A | 1.36 |
| Nerve | 24.0 | 2.0 | 0.9 | 1.36 |
| PDMS | 1.4 | 0.1 | 1 | 1.43 |
| Optical | 0** | 0** | N/A | 1.47 |
| Fiber | | | | |
| Core | | | | |
| Optical | 0** | 0** | N/A | 1.42 |
| Fiber | | | | |
| Cladding | | | | |

* Directly from the supplier: Ocean Insight FL, USA
**Approximated to be zero to simplify the calculation

### Results

### Nerve's Thermal Exposure was Similar Between INI and Resistive Heating

To compare the changes in the electrophysiological responses induced by the two heating modalities, it is necessary to examine if there is a systematic bias in the data availability and thermal exposure between the two heating modalities. From all six tested nerves, 1927 CAPs were collected during INI, and 2260 CAPs were collected during resistive heating. A paired t-test of the number of valid CAPs in each nerve between the two heating modalities did not show a significant difference (p=0.70). The ΔT step from one heating trial to the next was 1.4±0.9° C. for INI and 1.3±0.8° C. for resistive heating with no significant difference (p=0.55). The total temperature elevation dose (calculated as the summation of ΔT×duration) was calculated for each heating modality across the six nerves. On average, a temperature elevation dose of 2405° C.·s was applied during INI and 2945° C. s was applied during resistive heating. A paired t-test of the temperature elevation doses in each nerve between the two heating modalities did not show a significant difference (p=0.17). Overall, the data availability and thermal exposure of the nerves were similar for both INI and resistive heating, allowing an unbiased comparison of the inhibitory effect.

### Resistive Heating can Induce a Selective Inhibition Effect Similar to INI

As the representative data in FIG. 9, elements a and b shows, resistive heating produced a selective inhibition effect, similar to INI. The ΔT for this representative data (INI: 9.5° C., RH: 10.6° C.) was high enough to induce a block on the slow-conducting subpopulations (shown to the right of the dotted line of FIG. 9 element a), while still too low to significantly inhibit the fast-conducting subpopulations (shown to the left of the dotted line of FIG. 9, element a). Raw normalized reactive area under the curve (RAUC) data (shown in FIG. 9, element b) confirmed that both heating modalities could induce a similar drop in the signal for the slow-conducting subpopulation. The control test conducted after the heating test showed a response similar to that of the initial control test, suggesting that the health of the nerve was not acutely impacted.

To further examine the effect on all tested nerves, the normalized inhibition strength for the fast- and slow-conducting subpopulations of CAPs recorded during the quasi-steady state of the heating period was calculated (see Methods). When comparing across different subpopulations, the normalized inhibition strength of the slow-conducting subpopulations was generally higher than the corresponding values for fast-conducting subpopulations from the same CAP, and the trends of resistive heating and INI are similar overall. When the inhibition probability was calculated for each 1° C., range, it increased as the temperature elevation increased, for both modalities. A probit regression was conducted for the slow-conducting subpopulation and estimated the threshold temperature elevation for 50% inhibition probability was 9.34° C. for INI and 10.02° C. for resistive heating. The probit regression was not conducted for the fast-conducting subpopulation as the test was not designed to induce a full block of the fast-conducting subpopulation, which would be required for a proper probit fit.

TABLE 3
Probit regression optimal fits for
the slow-conducting subpopulation

| | | | Root |
|---|---|---|---|
| | | | mean |
| Heating | | | square |
| Modality | T₅₀ | δ | d error |
| IR Neural | 9.34 | 1.59 | 0.032 |
| Inhibition | [9.17, 9.50] | [1.35, 1.82] | |
| Resistive | 10.02 | 2.261 | 0.081 |
| heating | [9.52, 10.52] | [1.56, 2.97] | |

Comparing the fitted parameters, the ΔT threshold for inhibiting the slow-conducting subpopulation with resistive heating (10.02° C.) was higher than the threshold with INI (9.34° C.). In summary, the results show that resistive heating can reproduce the selective inhibition effect on a slow-conducting small-diameter subpopulation with a higher temperature threshold. Ranges are indicated within brackets for each of the mean values shown.

### Resistive Heating can Induce Selective Inhibition Reliably Across Nerves, With a Lower Power Requirement

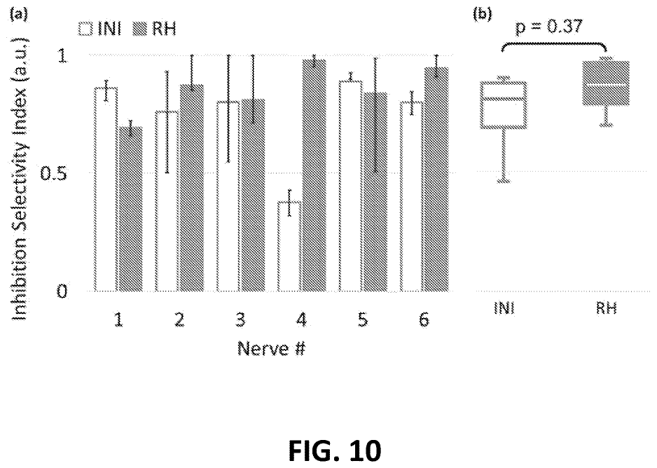

The inhibition selectivity index was calculated when an inhibition event was present for either or both subpopulations. The calculation was conducted for each nerve separately, across the whole tested temperature range. As shown in FIG. 10, resistive heating had a higher average inhibition selectivity index (0.76) than INI (0.86), although the difference was not significant according to a paired t-test (p=0.37). Since the inhibition selectivity index for both methods were higher than 0.5. As shown in FIG. 10, the variance in the inhibition selectivity index across different nerves was smaller for resistive heating. This suggests that resistive heating can induce selective inhibition more reliably when the nerve's geometry and fascicle orientation varies from one nerve to another.

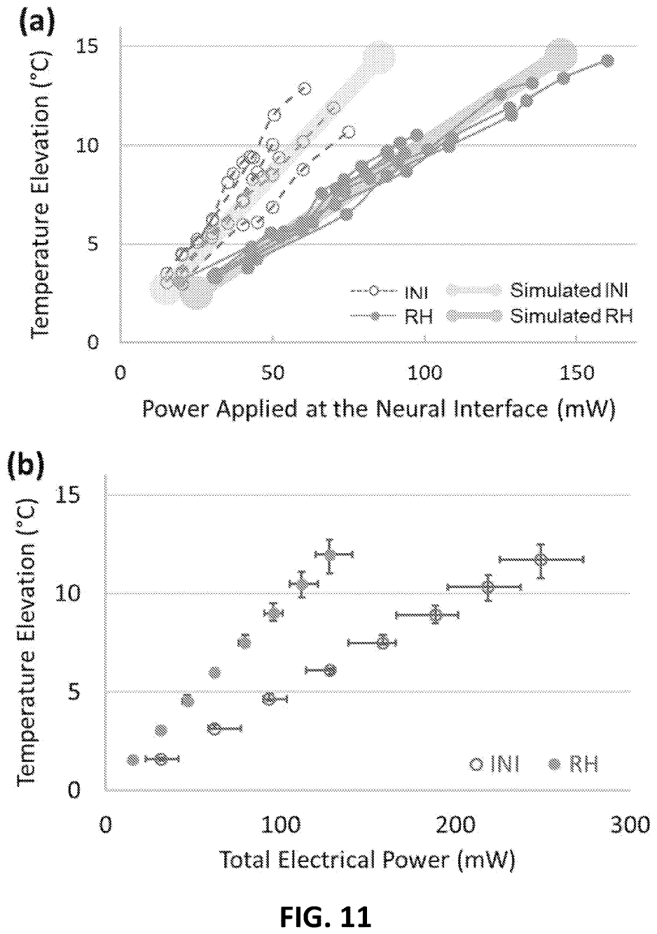

The efficiency of power applied at the neural interface indicates how efficiently the power was coupled to the nerve. For INI, the power applied at the neural interface is the optical power emitted from the optical fiber. For resistive heating, the power applied at the neural interface is the electrical power applied to the heating wire. To compare the efficiency of power applied at the neural interface between the two heating modalities, the final ΔT was calculated as the average ΔT during the last three seconds of the heating period, when a given power was applied at the neural interface. FIG. 11, element a, shows the power applied at the neural interface vs. the final ΔT for each heating modality across different nerves. INI was more effective in inducing ΔT when a given power was applied at the neural interface. However, the ΔT induced by resistive heating was more repeatable across the six tested nerves. INI exhibited a larger variance in the ΔT as the power applied at the neural interface increased. Using linear regression, to achieve an inhibition probability of 50% on the slow-conducting subpopulation, INI needs to apply 52.4 mW of power at the neural interface for its T₅₀ at 9.34° C., whereas resistive heating needs to apply 103.1 mW power at the neural interface for its T₅₀ at 10.02° C.

The efficiency of total electrical power is another critical aspect for implementing selective inhibition using an implantable design. The total electrical power consumption of the two modalities and their corresponding final ΔT values were measured. Linear regression was conducted to calculate the total electrical power required to achieve a given target temperature and vice versa. The predicted values and prediction intervals are shown in FIG. 11, element b. As shown in FIG. 11, element b, resistive heating has higher overall energy efficiency as it only required 49.7% of the total electrical power required by INI to achieve the same ΔT. The prediction intervals were smaller for resistive heating than that for INI as resistive heating is more repeatable across different nerves. According to the tests, the conversion efficiency from total electrical power to the power applied at the neural interface for the current design of resistive heating cuff was 93%, whereas that of the laser diode used in this experiment was only 24-27% depending on the optical power. Hence, to achieve an inhibition probability of 50% on the slow-conducting subpopulation, INI needs 197.9 mW for its T₅₀ at 9.34° C. whereas resistive heating only needs 106.8 mW for its T₅₀ at 10.02° C. Therefore, the current implementation of resistive heating can achieve the same 50% probability of inhibition with half of the total electrical power required by INI.

Resistive Heating is Selective and Uses with Less Total Electrical Power.

The nerve cuff design with resistive heating can induce the same size-selective inhibition as INI but with less total electrical power used (see FIG. 11, element b). Additionally, the power-to-ΔT relationship is more predictable for resistive heating (see FIG. 11), and size selectivity of nerve fibers is more reliable using resistive heating.

Resistive heating and INI heat the nerve fibers in different ways. The simulation models helped to deduce the ΔT at the axon region of the nerve based on ΔT surface measurements. For INI, volumetric heating is generated directly inside the nerve as IR light is absorbed by the whole nerve. Hence, the core temperature of the nerve during INI is higher than the measured ΔT. For resistive heating, heat is generated in the heating cuff and then diffuses into the nerve based on heat conduction. The core temperature of the nerve during resistive heating is, therefore, lower than the measured ΔT value at the nerve surface.

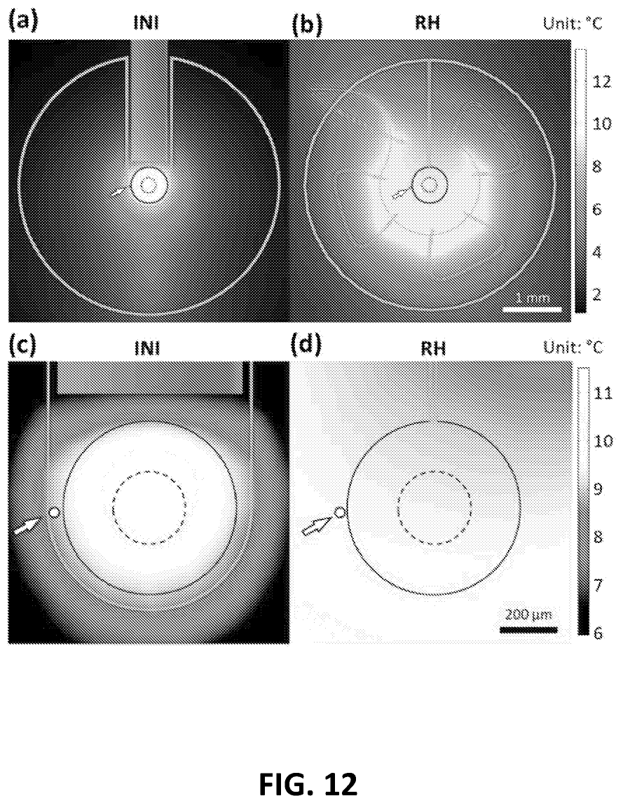

In practice, IR light of INI is incident on one side of a nerve, therefore forming a thermal gradient across the cross-section of the nerve (see FIG. 12, element a). The nerve region distal to the optical fiber tip has a lower ΔT because the light is strongly absorbed in the proximal region. Therefore, when the ΔT in the proximal region is high enough to induce inhibition (“hot side”), the subpopulation located distally may not reach the ΔT threshold yet (“cold side”). If the large-diameter subpopulation is predominantly located at the proximal end (“hot side”) and the small-diameter subpopulation is predominantly located at the distal end (“cold side”), the large-diameter subpopulation may be inhibited firstly. In other words, the variance in size selectivity observed in the INI experiment results can be a manifestation of spatial specificity. A previous study has shown that the high spatial specificity of IR light delivery can cause variance in the results of IR neuromodulation.

In contrast resistive heating showed a reliable size-selective inhibition when different nerves were tested. The resistive heating is more reliable because the resistance-heating nerve cuff design has a heating wire surrounding the nerve, inducing a more uniform temperature elevation across the cross-section of the nerve (see FIG. 12, element b). The nerve cuff design also minimized the possibility of losing size selectivity due to a shift in the orientation of the heating cuff after implantation, which can happen with the optical fiber used for INI.

Resistive heating using the nerve cuff design was also thermally safer than INI and less prone to movement or damage. For a normal human body temperature of 37° C., resistive heating can require a temperature threshold of approximately 40° C. to selectively inhibit small-diameter axons. This threshold temperature is lower than the recognized threshold that may cause acute damage to the nerve when exposed in the short term. Also, the modeling described above indicates that resistive heating generates a spatially homogeneous temperature elevation across the nerve (see FIG. 12, element d), minimizing the danger of local overheating due to spatial thermal gradient during INI (see FIG. 12, element c). The nerve cuff design using resistive heating can be less prone to movement following chronic implantation due to the 360° wrapping design than a partially or fully implanted optical fiber for chronic INI. Additionally, the nerve cuff can be composed of materials with mechanical compliance near or matching with nearby tissue to minimize deleterious mechanical effects and associated foreign body responses as compared to current INI systems. Finally, it is also important to note that the resistive heating cuff does not inject charge into the tissue, as standard electrical stimulation and block methods do, and therefore will not induce charge-injection-related tissue damage either.

From the above description, those skilled in the art will perceive improvements, changes, and modifications. Such improvements, changes and modifications are within the skill of one in the art and are intended to be covered by the appended claims.

The following is claimed:

1. A system comprising:
a controller device configured to deliver an amount of energy to a neural prosthetic device; and
the neural prosthetic device configured to be positioned near at least a part of a nerve of a patient, the neural prosthetic device comprising:
a heat delivery component configured to convert the amount of energy to an amount of heat and to deliver the amount of heat to the part of the nerve to block conduction in at least a portion of the nerve; and
a temperature sensor configured to record a temperature of the nerve; and the controller device further configured to:
store a thermal dose profile for the patient and information regarding a predetermined heat boundary for the nerve; and
alter the amount of energy sent to the neural prosthetic device based on the temperature of the nerve to maintain the temperature of the nerve within the thermal dose protocol of the patient and the predetermined heat boundary for the nerve.

2. The system of claim 1, wherein the neural prosthetic device further comprises one or more recording electrodes configured to detect neural signals indicative of pain, and
the controller device is further configured to:
receive the neural signals indicative of pain;
determine the patient's level of pain; and
alter the amount of energy sent to the heat delivery component based on the patient's level of pain.

3. The system of claim 1, further comprising a thermal insulation structure surrounding at least a portion of the heat delivery component to reduce heat dissipation.

4. The system of claim 1, wherein the heat delivery component comprises at least one resistive heating element, ultrasound heating element, or radio-frequency heating element.

5. The system of claim 1, wherein the heat delivery component comprises a plurality of heating elements spread around the nerve and/or along the nerve.

6. The system of claim 5, wherein the controller device is configured to select one or more of the plurality of heating elements to control a spatial location where the amount of heat is delivered.

7. The system of claim 1, wherein the controller device comprises a failure detection mechanism configured to detect a fault, wherein the fault occurs if an integrity of at least a portion of the neural prosthetic device being compromised, causing an internal electric circuit to be exposed.

8. The system of claim 7, wherein the controller device is further configured to cut power to at least the heat delivery component of the neural prosthetic device when the failure detection mechanism detects the fault.

9. The system of claim 1, wherein the neural prosthetic device further comprises a nerve cuff having an inner side configured to face the nerve, an outer side, and at least the heat delivery component sandwiched between the inner and outer sides.

10. The system of claim 9, wherein the temperature sensor is a thermocouple positioned on the inner side of the heat delivery component facing the nerve.

11. The system of claim 1, wherein the thermal dose profile comprises a desired temperature elevation times a desired heating period, wherein a temperature change determines strength and/or size selectivity of block of a set of one or more fibers in the nerve.

12. The system of claim 1, further comprising a power delivery mechanism to deliver power from an external power source to the controller device.

13. A method comprising:
determining, by a controller, an amount of energy to be delivered to a heat delivery component of a neural prosthetic device for conversion to a heat signal and application to a nerve to block conduction in at least a portion of the nerve, wherein the amount of energy to be delivered is determined based on a thermal dose profile stored by the controller;
receiving, by the controller, a signal comprising a temperature of the nerve when the heat signal is applied to the nerve; and
altering, by the controller, the amount of energy received by the heat delivery component based on the temperature of the nerve so the amount of energy follows the thermal dose protocol and the temperature of the nerve remains within an allowable range.

14. The method of claim 13, wherein the conduction is blocked in at least the portion of the nerve to treat pain.

15. The method of claim 13, further comprising detecting, by the controller, a fault, wherein the fault occurs when an integrity of at least a portion of the neural prosthetic device being compromised, causing an internal electric circuit to be exposed

16. The method of claim 13, wherein the signal is delivered to the controller by a temperature measurement component of the neural prosthetic device.

17. The method of claim 13, further comprising receiving, by the controller, an input selecting at least one fiber in the nerve for treatment.

18. The method of claim 13, further comprising receiving, by the controller, an indication of pain from one or more electrophysiological recording electrodes configured to detect pain signals from the nerve.

19. The method of claim 13, further comprising:
receiving, by the controller, power from an external source; and
delivering, by the controller, at least a portion of the power to the neural prosthetic device.

20. The method of claim 13, further comprising selecting, by the controller, one or more axons to receive the heat signal based on temperature change.
